(19) United States
(12) Patent Application Publication
(10) Pub. No.: US 2026/0337185 A1
(43) Pub. Date: Oct. 29, 2026

(54) SOLID STATE FORMS OF CILOFEXOR SALTS

(71) Applicant: ASSIA CHEMICAL INDUSTRIES LTD., Tel Aviv (IL)

(72) Inventors: Anantha Rajmohan Muthusamy, Sivakasi, Tamil Nadu (IN); Amit Singh, Greater Noida, Uttar Pradesh (IN); Azhaguraja S, Dist. Dindigul, Tamil Nadu (IN)

(21) Appl. No.: 19/114,918

(22) PCT Filed: Oct. 23, 2023
(86) PCT No.: PCT/IB2023/060680

§ 371 Date: Mar. 25, 2025

(30) Foreign Application Priority Data
Oct. 25, 2022 (IN) 202211060814
Dec. 12, 2022 (IN) 202211071728

(51) Int. Cl. C07D 413/14

(52) U.S. Cl. CPC C07D 413/14

(57) ABSTRACT

The present disclosure encompasses solid state forms of Cilofexor and salts thereof, in embodiments crystalline polymorphs of Cilofexor and salts thereof, processes for preparation thereof, and pharmaceutical compositions thereof.

### FIELD OF THE DISCLOSURE

The present disclosure encompasses solid state forms of Cilofexor and salts thereof, in embodiments crystalline polymorphs of Cilofexor and salts thereof, processes for preparation thereof, and pharmaceutical compositions thereof.

### BACKGROUND OF THE DISCLOSURE

Cilofexor, 2-[3-[2-chloro-4-[[5-cyclopropyl-3-(2,6-dichlorophenyl)-1,2-oxazol-4-yl]methoxy]phenyl]-3-hydroxyazetidin-1-yl]pyridine-4-carboxylic acid, has the following chemical structure:

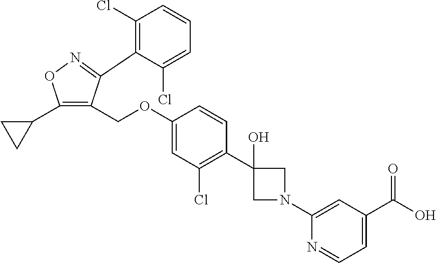 [SMILES: O=C(O)c1ccnc(N2CC(O)(c3ccc(OCc4c(-c5c(Cl)cccc5Cl)noc4C4CC4)cc3Cl)C2)c1]

Cilofexor is reported to be FXR agonist that binds to the NR1H4 receptor (FXR) and acts as agonist or modulator of FXR. Cilofexor is under clinical investigation for the treatment and/or prophylaxis of diseases and conditions through binding of the NR1H4 receptor, for example liver disease, such as nonalcoholic steatohepatitis (“NASH”), primary biliary cholangitis/cirrhosis (PBC) and primary sclerosing cholangitis.

The compound is described in International Publication No. WO2013/007387.

Salts and solid forms are described in International Publication No. WO2020/172075.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms of Cilofexor.

### SUMMARY OF THE DISCLOSURE

The present disclosure provides solid state forms of Cilofexor and salts thereof, processes for preparation thereof, and pharmaceutical compositions thereof. Preferably, according to any aspect or embodiment of the disclosure, the Cilofexor salt is a tromethamine salt. Accordingly, in a first aspect, there is provided crystalline Cilofexor: Tromethamine, which is a salt. In a further aspect, there is provided solid state forms of Cilofexor:Tromethamine. These solid state forms can be used to prepare other solid state forms of Cilofexor, Cilofexor salts and their solid state forms.

The present disclosure also provides uses of the said solid state forms of Cilofexor and salts thereof in the preparation of other solid state forms of Cilofexor or salts thereof.

The present disclosure provides solid state forms of Cilofexor and salts thereof for use in medicine, including for the treatment of liver disease, such as nonalcoholic steatohepatitis (“NASH”), primary biliary cholangitis/cirrhosis (PBC) and primary sclerosing cholangitis.

The present disclosure also encompasses the use of solid state forms of Cilofexor and salts thereof of the present disclosure for the preparation of pharmaceutical compositions and/or formulations.

In another aspect, the present disclosure provides pharmaceutical compositions comprising solid state forms of Cilofexor particularly Cilofexor Tromethamine, according to the present disclosure.

The present disclosure includes processes for preparing the above mentioned pharmaceutical compositions. The processes include combining any one or a combination of the solid state forms of Cilofexor, particularly Cilofexor Tromethamine, of the present disclosure with at least one pharmaceutically acceptable excipient.

The solid state forms of Cilofexor, particularly Cilofexor Tromethamine, as defined herein and the pharmaceutical compositions or formulations of the solid state forms of Cilofexor, particularly Cilofexor Tromethamine, of the present disclosure may be used as medicaments, such as for the treatment of liver disease, Primary sclerosing cholangitis, Non-alcoholic fatty liver disease; Non-alcoholic steatohepatitis and Primary biliary cirrhosis.

The present disclosure also provides methods of treating liver disease, Primary sclerosing cholangitis, Non-alcoholic fatty liver disease; Non-alcoholic steatohepatitis and Primary biliary cirrhosis, by administering a therapeutically effective amount of any one or a combination of the solid state forms of Cilofexor, particularly Cilofexor Tromethamine, of the present disclosure, or at least one of the above pharmaceutical compositions, to a subject suffering from liver disease, Primary sclerosing cholangitis, Non-alcoholic fatty liver disease; Non-alcoholic steatohepatitis and Primary biliary cirrhosis or otherwise in need of the treatment.

The present disclosure also provides uses of the solid state forms of Cilofexor, particularly Cilofexor Tromethamine, of the present disclosure, or at least one of the above pharmaceutical compositions, for the manufacture of medicaments for treating liver disease, Primary sclerosing cholangitis, Non-alcoholic fatty liver disease; Non-alcoholic steatohepatitis and Primary biliary cirrhosis.

### BRIEF DESCRIPTION OF THE DRAWINGS

FIG. 1 shows a characteristic X-ray powder diffraction pattern (XRPD) of Cilofexor Tromethamine Form CT1.

FIG. 2 shows a characteristic XRPD of Cilofexor Tromethamine Form CT2.

FIG. 3 shows a characteristic XRPD of Cilofexor Tromethamine Form CT3.

FIG. 4 shows a characteristic XRPD of Cilofexor Tromethamine Form CT4.

FIG. 5 shows a characteristic XRPD of Cilofexor Tromethamine Form CT5.

FIG. 6 shows a characteristic XRPD of amorphous Cilofexor Tromethamine.

FIG. 7a shows solid state ¹³C NMR spectrum of Cilofexor Tromethamine Form CT1 (full scan).

FIG. 7b shows solid state ¹³C NMR spectrum of Cilofexor Tromethamine Form CT1 (at the range of 0-100 ppm).

FIG. 7c shows solid state ¹³C NMR spectrum of Cilofexor Tromethamine Form CT1 (at the range of 100-200 ppm).

FIG. 8a shows solid state ¹³C NMR spectrum of Cilofexor Tromethamine Form CT3 (full scan).

FIG. 8b shows solid state ¹³C NMR spectrum of Cilofexor Tromethamine Form CT3 (at the range of 0-100 ppm).

FIG. 8c shows solid state ¹³C NMR spectrum of Cilofexor Tromethamine Form CT3 (at the range of 100-200 ppm).

### DETAILED DESCRIPTION OF THE DISCLOSURE

The present disclosure encompasses solid state forms of Cilofexor and salts thereof, in embodiments crystalline polymorphs of Cilofexor and salts thereof, processes for preparation thereof, and pharmaceutical compositions thereof. According to any aspect or embodiment of the disclosure, the Cilofexor is Cilofexor Tromethamine. In embodiments, the present disclosure provides crystalline forms of Cilofexor Tromethamine designated as Form CT1, Form CT2, Form CT3, Form CT4 and Form CT5 (defined herein).

Solid state properties of Cilofexor, and crystalline polymorphs thereof can be influenced by controlling the conditions under which Cilofexor and crystalline polymorphs thereof are obtained in solid form.

A solid state form (or polymorph) of Cilofexor and salts thereof, particularly Cilofexor Tromethamine as described in any aspect or embodiment of the present disclosure may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Cilofexor, particularly Cilofexor Tromethamine, described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Cilofexor, particularly Cilofexor Tromethamine. In some embodiments of the disclosure, the described crystalline polymorph of Cilofexor, particularly Cilofexor Tromethamine, may contain from about 1% to about 20% (w/w), from about 5% to about 20% (w/w), or from about 5% to about 10% (w/w) of one or more other crystalline polymorph of the same Cilofexor, particularly Cilofexor Tromethamine. For example, a crystalline polymorph of Cilofexor and salts thereof, particularly Cilofexor Tromethamine according to any aspect or embodiment of the present invention may be polymorphically pure, and may contain: about 20% (w/w) or less, about 10% (w/w) or less, about 5% (w/w) or less, about 2% (w/w) or less, about 1% (w/w) or less, or about 0% of any other forms of Cilofexor and salts thereof, particularly Cilofexor Tromethamine, as measured, for example, by XRPD. Thus, for example, a crystalline polymorph of Cilofexor Tromethamine as described in any aspect or embodiment herein, which is polymorphically pure, may contain: about 20% (w/w) or less, about 10% (w/w) or less, about 5% (w/w) or less, about 2% (w/w) or less, about 1% (w/w) or less, or about 0% of any other forms of Cilofexor and salts thereof, particularly Cilofexor Tromethamine. As another example, a crystalline polymorph of Cilofexor and salts thereof, particularly Cilofexor Tromethamine as described in any aspect or embodiment herein, which is polymorphically pure, may contain: about 20% (w/w) or less, about 10% (w/w) or less, about 5% (w/w) or less, about 2% (w/w) or less, about 1% (w/w) or less, or about 0% of any other forms of the Cilofexor and salts thereof, particularly Cilofexor Tromethamine. Thus, a crystalline polymorph of Cilofexor Tromethamine as described in any aspect or embodiment herein, which is polymorphically pure, may contain: about 20% (w/w) or less, about 10% (w/w) or less, about 5% (w/w) or less, about 2% (w/w) or less, about 1% (w/w) or less, or about 0% of any other forms of Cilofexor Tromethamine. Alternatively, a crystalline polymorph of Cilofexor and salts thereof, particularly Cilofexor Tromethamine according to any aspect or embodiment of the present invention may be polymorphically pure and may contain greater than about 80% (w/w), greater than about 90% (w/w), greater than about 95% (w/w), greater than about 98% (w/w), greater than about 99% (w/w), or about 100% of the crystalline polymorph of Cilofexor and salts thereof, particularly Cilofexor Tromethamine.

The solid state forms of Cilofexor and salts thereof, particularly Cilofexor Tromethamine, as described in any aspect or embodiment of the present disclosure may be chemically pure, or substantially free of any other compounds.

A compound may be referred to herein as chemically pure or purified compound or as substantially free of any other compounds. As used herein, the terms “chemically pure” or “purified” or “substantially free of any other compounds” refer to a compound that is substantially free of any impurities including enantiomers of the subject compound, diastereomers or other isomers. A chemically pure or purified compound or a compound that is substantially free of any other compound will be understood to mean that it contains about 10% (w/w) or less, about 5% (w/w) or less, about 4% (w/w) or less, about 3% (w/w) or less, about 2% (w/w) or less, about 1.5% (w/w) or less, about 1% (w/w), about 0.8% (w/w) or less, about 0.6% (w/w) or less about 0.4% (w/w) or less about 0.2% (w/w) or less or less, about 0.1% (w/w) or less or about 0% of any other compound as measured, for example, by HPLC. Alternatively, A chemically pure or purified compound or a compound that is substantially free of any other compound will be understood to mean that it contains about 10% area percent or less, about 5% area percent or less, about 4% area percent or less, about 3% area percent or less, about 2% area percent or less, about 1.5% area percent or less, about 1% area percent or less, about 0.8% area percent or less, about 0.6% area percent or less, about 0.4% area percent or less, about 0.2% area percent or less, about 0.1% area percent or less, or about 0% of any other compound as measured by HPLC. Thus, pure or purified Cilofexor and salts thereof, particularly Cilofexor Tromethamine, described herein as substantially free of any compounds would be understood to contain greater than about 90% (w/w), greater than about 95% (w/w), greater than about 96% (w/w), greater than about 97% (w/w), greater than about 98% (w/w), greater than about 98.5% (w/w), greater than about 99% (w/w), greater than about 99.2%, (w/w) greater than about 99.4% (w/w), greater than about 99.6% (w/w), greater than about 99.8% (w/w), greater than about 99.9% (w/w), or about 100% of the subject Cilofexor, particularly Cilofexor Tromethamine. Thus, Cilofexor Tromethamine, described herein as substantially free of any compounds would be understood to contain greater than about 90% (w/w), greater than about 95% (w/w), greater than about 96% (w/w), greater than about 97% (w/w), greater than about 98% (w/w), greater than about 98.5% (w/w), greater than about 99% (w/w), greater than about 99.2%, (w/w) greater than about 99.4% (w/w), greater than about 99.6% (w/w), greater than about 99.8% (w/w), greater than about 99.9% (w/w), or about 100% of Cilofexor Tromethamine. Alternatively, pure or purified Cilofexor and salts thereof, particularly Cilofexor Tromethamine, described herein as substantially free of any compounds would be understood to contain greater than about 90% area percent, greater than about 95% area percent, greater than about 96% area percent, greater than about 97% area percent, greater than about 98% area percent, greater than about 98.5% area percent, greater than about 99% area percent, greater than about 99.2%, area percent, greater than about 99.4% area percent, greater than about 99.6% area percent, greater than about 99.8% area percent, greater than about 99.9% area percent, or about 100% of the subject Cilofexor, particularly Cilofexor Tromethamine. Thus, Cilofexor Tromethamine, described herein as substantially free of any compounds would be understood to contain greater than about 90% area percent, greater than about 95% area percent, greater than about 96% area percent, greater than about 97% area percent, greater than about 98% area percent, greater than about 98.5% area percent, greater than about 99% area percent, greater than about 99.2%, area percent, greater than about 99.4% area percent, greater than about 99.6% area percent, greater than about 99.8% area percent, greater than about 99.9% area percent, or about 100% of Cilofexor Tromethamine.

Depending on which other crystalline polymorphs a comparison is made, the crystalline polymorphs of Cilofexor, particularly Cilofexor Tromethamine, of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Cilofexor, particularly Cilofexor Tromethamine, referred to herein as being characterized by graphical data “as depicted in” or “as substantially depicted in” a Figure will thus be understood to include any crystal forms of Cilofexor, particularly Cilofexor Tromethamine, characterized with the graphical data having such small variations, as are well known to the skilled person, in comparison with the Figure.

As used herein, crystalline Cilofexor:Tromethamine is a distinct molecular species. Crystalline Cilofexor:Tromethamine may be a co-crystal of Cilofexor and Tromethamine. Alternatively crystalline Cilofexor:Tromethamine may be a salt.

“Co-Crystal” or “Cocrystal” as used herein is defined as a crystalline material including two or more molecules in the same crystalline lattice and associated by non-ionic and non-covalent bonds. In some embodiments, the cocrystal includes two molecules which are in natural state.

“Cocrystal former” or “crystal former” as used herein is defined as a molecule that forms a cocrystal with Cilofexor or salts thereof, for example Tromethamine.

As used herein, solid state forms or crystalline polymorphs or crystal forms of Cilofexor relates to solid state forms of Cilofexor and Cilofexor salts, particularly Cilofexor Tromethamine, and include co-crystal forms of Cilofexor (and salts thereof) and a crystal former.

As used herein, and unless stated otherwise, the term “anhydrous” in relation to crystalline forms of Cilofexor, particularly Cilofexor Tromethamine, relates to a crystalline form of Cilofexor, particularly Cilofexor Tromethamine, which does not include any crystalline water (or other solvents) in a defined, stoichiometric amount within the crystal. Moreover, an “anhydrous” form would generally not contain more than 1% (w/w), or not more than 0.8% (w/w), or not more than 0.6% (w/w), or not more than 0.4% (w/w), or not more than 0.2% (w/w) of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Cilofexor, particularly Cilofexor Tromethamine, of the present disclosure corresponds to a crystalline polymorph of Cilofexor, particularly Cilofexor Tromethamine, that is physically separated from the reaction mixture in which it is formed.

As used herein, unless stated otherwise, the XRPD measurements are taken using copper Kα radiation wavelength 1.54187 Å. XRPD peaks reported herein are measured using CuKα radiation, X=1.54187 Å, typically at a temperature of 25±3° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, unless stated otherwise, solid state ¹³C NMR data is obtained using ¹³C CP/MAS NMR method. Particularly, as used herein, unless stated otherwise, the ¹³C CP/MAS NMR reported herein are measured at 500 MHz, preferably at a temperature of at 298 K±3° C.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

The present disclosure includes a crystalline form of Cilofexor: Tromethamine. Crystalline Cilofexor:Tromethamine may be a co-crystal of Cilofexor and Tromethamine. Alternatively, crystalline Cilofexor:Tromethamine may be a salt, i.e., Cilofexor Tromethamine. Cilofexor:Tromethamine may be in a ratio between about 2:1 to about 1:2 of Cilofexor and Tromethamine. In one embodiment, Cilofexor: Tromethamine may be in a 1:1 ratio of Cilofexor and Tromethamine. In one embodiment, Cilofexor:Tromethamine may be in a 1:1 ratio of Cilofexor and Tromethamine. Particularly, according to any aspect or embodiment of the disclosure, Cilofexor: Tromethamine may be in a ratio of 1:1 moles of Cilofexor and Tromethamine.

Preferably, crystalline Cilofexor:Tromethamine according to any aspect or embodiment of the disclosure is salt i.e. Cilofexor Tromethamine. More preferably, crystalline Cilofexor:Tromethamine according to any aspect or embodiment of the disclosure is a salt, wherein the Cilofexor and Tromethamine are present in a molar ratio of 1:1.

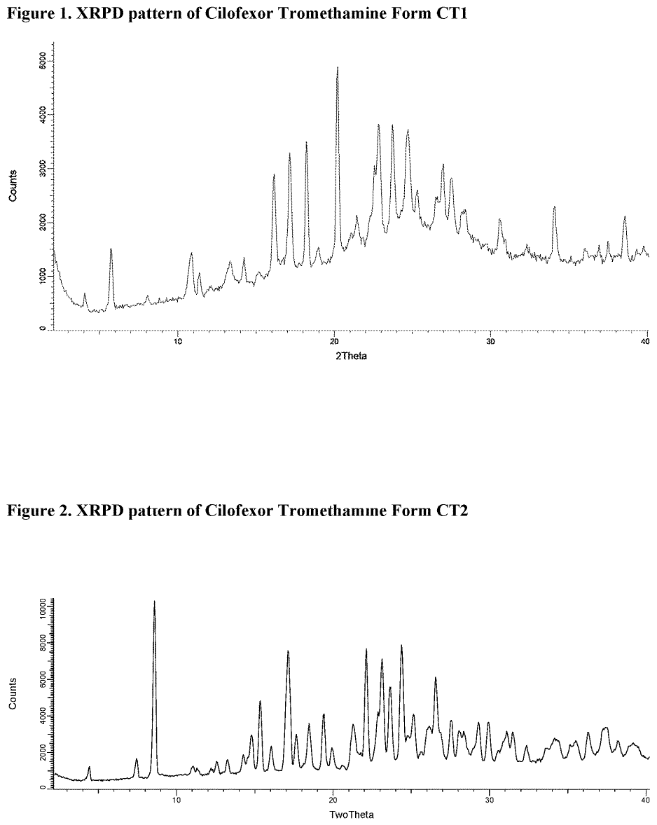

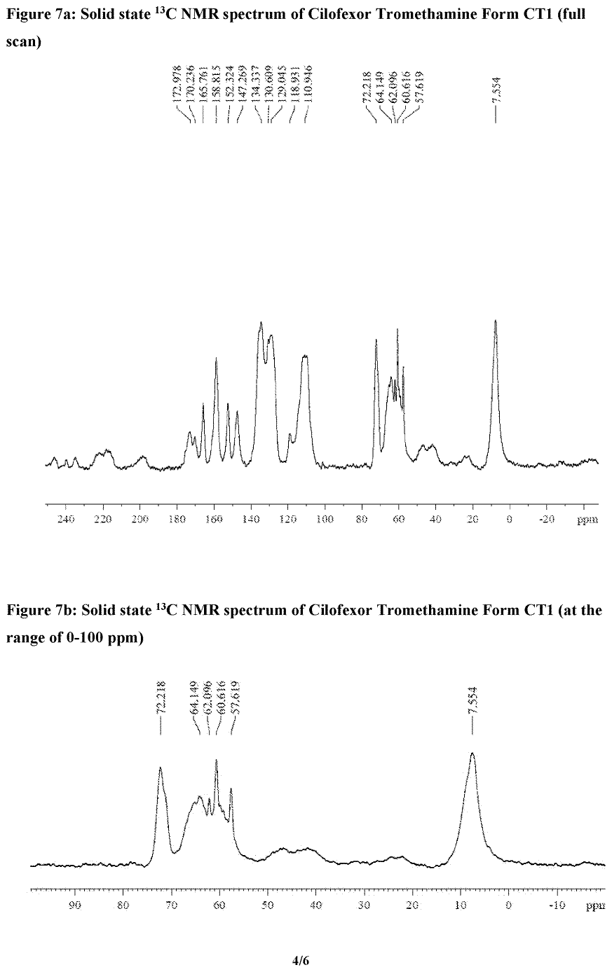

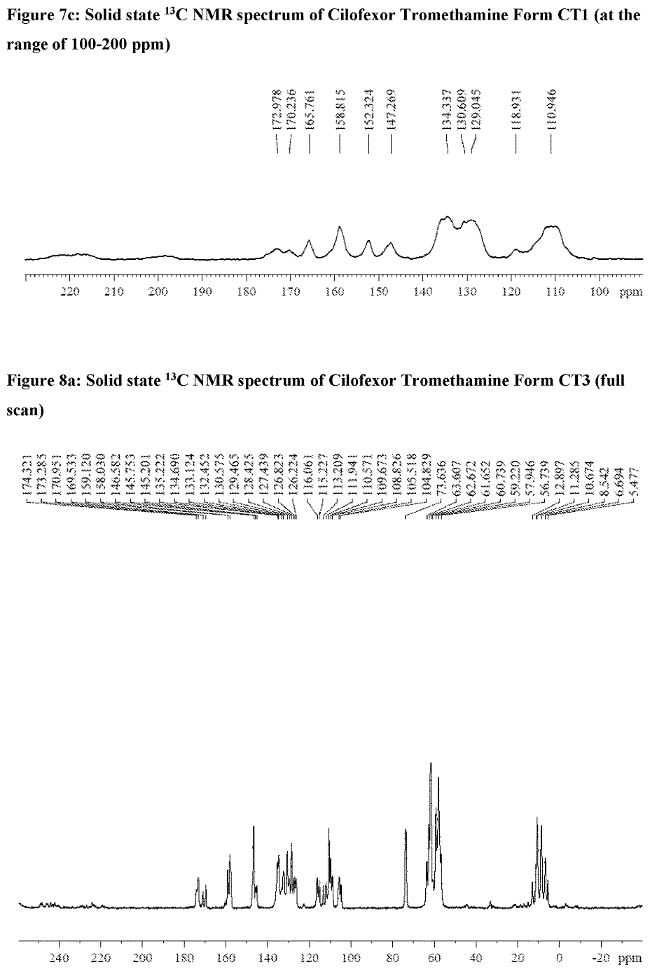

The present disclosure includes a crystalline polymorph of Cilofexor: Tromethamine, designated Form CT1. The crystalline Form CT1 of Cilofexor: Tromethamine may be characterized by data selected from one or more of the following: an X-ray powder diffraction pattern substantially as depicted in FIG. 1; an X-ray powder diffraction pattern having peaks at 5.7, 16.1, 20.2, 21.5 and 23.8 degrees 2-theta±0.2 degrees 2-theta; a solid state ¹³C NMR spectrum having peaks at 57.6, 111.0, 129.1, 147.3, 158.8 and 165.8 ppm±0.2 ppm; a solid state ¹³C NMR spectrum having the following chemical shift absolute differences from a reference peak at 173.0 ppm±2 ppm of 115.4, 62.0, 43.9, 25.7, 14.2 and 7.2 ppm±0.1 ppm; a solid state ¹³C NMR spectrum substantially as depicted in FIGS. 7a, 7b or 7c; and combinations of these data.

Crystalline Form CT1 of Cilofexor:Tromethamine may be further characterized by an X-ray powder diffraction pattern having peaks at 5.7, 16.1, 20.2, 21.5 and 23.8 degrees 2-theta±0.2 degrees 2-theta, and also having any one, two, three, or four additional peaks selected from 10.8, 17.2, 18.2 and 34.1 degrees 2-theta±0.2 degrees 2-theta.

In any aspect or embodiment of the disclosure, crystalline Form CT1 of Cilofexor:Tromethamine may be characterized by any of the above data, and additionally having an absence of peaks at 4.5-5.2 degrees 2-theta±0.2 degrees 2-theta; and/or an absence of peaks at 8.6-9.9 degrees 2-theta±0.2 degrees 2-theta.

In one embodiment of the present disclosure, crystalline Form CT1 of Cilofexor:Tromethamine is isolated. Particularly, according to any aspect or embodiment of the disclosure, crystalline Form CT1 of Cilofexor:Tromethamine is isolated.

The molar ratio between Cilofexor and Tromethamine may be 1:1. Thus, according to any aspect or embodiment of the disclosure, the molar ratio of Cilofexor and Tromethamine in crystalline Form CT1 of Cilofexor:Tromethamine is 1:1.

In one embodiment of the present disclosure, crystalline Form CT1 of Cilofexor:Tromethamine may be an anhydrous form. Particularly, according to any aspect or embodiment of the disclosure, Form CT1 of Cilofexor:Tromethamine is an anhydrous form.

Crystalline Form CT1 of Cilofexor:Tromethamine may be characterized by an X-ray powder diffraction pattern having peaks at 5.7, 10.8, 16.1, 17.2, 18.2, 20.2, 21.5, 23.8 and 34.1 degrees 2-theta±0.2 degrees 2-theta.

According to any aspect or embodiment of the disclosure, the crystalline Form CT1 of Cilofexor:Tromethamine may be polymorphically pure. The crystalline Form CT1 of Cilofexor:Tromethamine according to any aspect or embodiment of the disclosure, may be chemically pure.

Crystalline Form CT1 of Cilofexor:Tromethamine may be characterized by each of the above characteristics alone/or by all possible combinations, e.g., an XRPD pattern having peaks at 5.7, 16.1, 20.2, 21.5 and 23.8 degrees 2-theta±0.2 degrees 2-theta; an XRPD pattern as depicted in FIG. 1, and combinations thereof.

The present disclosure further provides a process for preparing crystalline Cilofexor:Tromethamine, preferably crystalline Form CT1 of Cilofexor:Tromethamine, as described in any aspect or embodiment of the disclosure.

The process may comprise crystallising Cilofexor:Tromethamine from either tetrahydrofuran (TIF) or a mixture of tetrahydrofuran and water. Particularly, the process may comprise:
(a) preparing a solution of Cilofexor:Tromethamine in either THF or a mixture of THF and water;
(b) crystallising the Cilofexor:Tromethamine from the solution;
(c) optionally isolating the crystals; and
(d) optionally drying to form Cilofexor:Tromethamine Form CT1.
   According to any aspect or embodiment of the process, the solution in step (a) may be prepared by combining Cilofexor, Tromethamine (i.e. TRIS or Tris(hydroxymethyl)-aminomethane), and either THF, or THF and water, to form a mixture, and preferably heating the mixture to obtain a solution. Alternatively, the solution in step (a) may be prepared by combining Cilofexor with THF, and combining with Tris(hydroxymethyl)-aminomethane. In an alternative embodiment, the solution in step (a) may be prepared by combining Cilofexor in THF with a solution of Tris(hydroxymethyl)aminomethane in water. In a further alternative embodiment, the solution in step (a) may be prepared by combining Cilofexor:Tromethamine with either THF, or THF and water. According to any aspect or embodiment of the process, the combining may be carried out at a temperature of: about 15° C. to about 30° C., about 18° C. to about 28° C., about 20° C. to about 25° C., particularly about 25° C. According to any aspect or embodiment of the process, the mixture in step (a) may be heated to a temperature of: about 40° C. to about 80° C., about 45° C. to about 70° C., about 45° C. to about 65° C., or about 50° C. to about 60° C. According to an aspect or embodiment of the process, the THF may be used in an amount of: about 5 ml to about 30 ml per gram of Cilofexor, about 8 ml to about 25 ml per gram of Cilofexor, about 10 ml to about 20 ml per gram of Cilofexor, about 12 ml to about 18 ml per gram of Cilofexor, or about 15 ml per gram of Cilofexor. In any embodiment of the process where the solution of Cilofexor:Tromethamine in step (a) is in THF and water, the water may be used in an amount of: about 0.1 ml to about 3 ml per gram of Cilofexor, about 0.2 ml to about 1.5 ml per gram of Cilofexor, about 0.3 ml to about 1.0 ml per gram of Cilofexor, about 0.3 ml to about 0.5 ml per gram of Cilofexor, or about 0.3 ml to about 0.4 ml per gram of Cilofexor. In embodiments where the solution of Cilofexor: Tromethamine in step (a) is in THF and water, the vol/vol ratio of water to THF may be in a range of: about 1:30 to about 1:60, about 1:35 to about 1:50, about 1:40 to about 1:45, or about 1:44. In any aspect or embodiment of the process, the Tromethamine may be used in an amount of: about 1.0 to about 2.0 mole equivalents relative to Cilofexor, about 1.0 to about 1.5 mole equivalents relative to Cilofexor, about 1.0 to about 1.2 mole equivalents relative to Cilofexor, about 1.0 to about 1.1 mole equivalents relative to Cilofexor, or about 1.1 mole equivalents relative to Cilofexor. According to any aspect or embodiment of the process, the solution in step (a), which is preferably heated to a temperature as discussed above, may be hot-filtered. Optionally, step (b) may comprise adding Form CT1 seed crystals to the mixture. When seed crystals are used, preferably the seeds are added in an amount of: about 25 mg to about 70 mg, about 30 mg to about 65 mg, or about 50 mg, per gram of Cilofexor. The heated mixture, with or without seeds, may be stirred at a temperature of: about 40° C. to about 80° C., about 45° C. to about 70° C., about 45° C. to about 65° C., or about 50° C. to about 60° C., preferably for about 2 hours to about 96 hours, about 4 hours to about 60 hours, about 6 hours to about 50 hours or about 8 hours to about 48 hours. Preferably, when seed crystals are added, the mixture may be stirred at a temperature of about 45° C. to 60° C., or about 50° C. to about 55° C., preferably for about 4 hours to about 12 hours, about 6 hours to about 10 hours, about 7 hours to about 9 hours, or about 8 hours. Preferably, when seed crystals are not used, the mixture may be stirred at a temperature of about 50° C. to 70° C., or about 55° C. to about 65° C., or about 60° C., preferably for about 20 hours to about 72 hours, about 30 hours to about 60 hours, about 36 hours to about 54 hours, or about 48 hours. According to any aspect or embodiment of the process, the resulting crystals may be isolated in step (c) by any suitable method, such as by filtration, decantation or centrifugation, preferably by filtration. According to any aspect or embodiment of the process, step (d) optionally or preferably According to any aspect or embodiment of the process, the crystals may be dried in step (d) to remove the solvent, for example to a constant weight. Optionally step (d) may comprise drying during suction filtration for a period of about 5 minutes to 2 hours, or about 10 minutes to about 1.5 hour, or about 10 minutes to about 30 minutes. According to any aspect or embodiment of the process, step (d) optionally, or preferably additionally, comprises drying the crystals at a temperature of: about 60° C. to about 100° C., about 65° C. to about 95° C., about 70° C. to about 90° C., about 75° C. to about 85° C., or about 80° C., preferably under reduced pressure or under vacuum. Preferably the drying can be carried out for a suitable period of time to remove the solvent, more preferably to a constant weight. According to any aspect or embodiment of the process, the drying may be carried out at a temperature of: about 70° C. to about 90° C., about 75° C. to about 85° C., or about 80° C., under reduced pressure or vacuum (more particularly in a vacuum tray dryer), preferably for about 8 hours to about 48 hours, about 10 hours to about 30 hours, about 12 hours to about 24 hours, about 16 hours to about 20 hours, or about 18 hours. Alternatively, according to any aspect or embodiment of the disclosed processes, the process may comprise:
(i) adding tetrahydrofuran to Cilofexor and Tris(hydroxymethyl)amino-methane;
(ii) heating the reaction mixture, and optionally stirring to form a solution; (iii) isolating Cilofexor:Tromethamine; and
(iv) drying.

According to any aspect or embodiment of the disclosed processes, in step (i), Tris(hydroxymethyl)aminomethane may be added in an amount of: about 1.2 molar equivalents to about 1 molar equivalents, or about 1.1 molar equivalents relative to Cilofexor.

According to any aspect or embodiment of the disclosed processes, in step (i), tetrahydrofuran may be added to Tris(hydroxymethyl)aminomethane and Cilofexor. Particularly, tetrahydrofuran may be added at an amount of: about 5 ml to about 30 ml, about 10 to about 25 ml, or about 15 ml, per gram of Cilofexor. Tetrahydrofuran may be added step (i) at temperature of: about 55° C. to about 40° C., or about 50° C. to about 35° C., or about 25° C.

In any aspect or embodiment of the disclosed processes, in step (ii), the reaction mixture may be heated to temperature of: about 80° C. to about 50° C., or about 70° C. to about 55° C., or about 60° C.

In any aspect or embodiment of the disclosed processes, in step (ii), the solution may be stirred at temperature of about 80° C. to about 50° C., or about 70° C. to about 55° C., or about 60° C., for a period of about 55 hours to about 65 hours, or about 52 hours to about 62 hours, or about 48 hours.

The process may further include isolating the obtained Cilofexor: Tromethamine [step (iii)], by any suitable procedure, such as filtration, decantation, or by centrifuge. Particularly the product may be isolated by vacuum filtration. Following isolation, the Cilofexor:Tromethamine is dried. The drying [step (iv)] may be carried out during, e.g. filtration for the period of about 10 minutes to 1 hour, or about 15 minutes to about 30 minutes. Optionally, and preferably additionally, the Cilofexor: Tromethamine is dried in a Vacuum tray dryer (VTD), typically at a temperature of about 40° C. to about 90° C., about 30° C. to about 85° C., or about 80° C. The drying may be carried out for any suitable time to remove the solvent (e.g. to a constant weight), typically about 25 hours to about 15 hour, or about 16 hours.

According to any aspect or embodiment of the disclosed processes, the process may alternatively comprise:
(i) adding tetrahydrofuran to Cilofexor;
(ii) heating the mixture, optionally with stirring;
(iii) adding a solution of Tris(hydroxymethyl)aminomethane in water to the mixture;
(iv) optionally adding seeds of form CT1 of Cilofexor:Tromethamine to the mixture;
(v) isolating the Cilofexor:Tromethamine; and
(vi) drying.

According to any aspect or embodiment of the disclosed processes, in step (i), tetrahydrofuran may be added to Cilofexor. Particularly, tetrahydrofuran may be added at an amount of: about 5 to about 30 ml, about 10 to about 25 ml, or about 15 ml of tetrahydrofuran per gram of Cilofexor. Tetrahydrofuran may be added in step (i) at temperature of: about 55° C. to about 40° C., or about 50° C. to about 35° C., or about 25° C.

In any aspect or embodiment of the disclosed processes, in step (ii), reaction mixture may be heated to temperature of: about 80° C. to about 45° C., or about 70° C. to about 50° C., or about 50° C. to about 55° C.

In any aspect or embodiment of the disclosed processes, in step (ii), the solution may be stirred at temperature of: about 80° C. to about 45° C., or about 70° C. to about 50° C., or about 50° C. to about 55° C., for a period of: about 5 minutes to about 40 minutes, about 10 minutes to about 30 minutes, or about 15 minutes.

Tris(hydroxymethyl)aminomethane in step (iii) may be dissolved at an amount of: about 0.85 to about 0.45 grams, or about 0.66 grams of Tris(hydroxymethyl)-aminomethane per ml of water. Tris(hydroxymethyl)aminomethane may be added at an amount of: about 0.20 to about 0.30 grams, or about 0.226 grams of Tris(hydroxymethyl)-aminomethane per gram of Cilofexor.

In any aspect or embodiment of the disclosed processes, in step (iii), the solution of Tris(hydroxymethyl)aminomethane in water may be added to the mixture in step (ii) at temperature of: about 80° C. to about 45° C., or about 70° C. to about 50° C., or about 50° C. to about 55° C.

In any aspect or embodiment of the disclosed processes, in step (iv), seeds of form CT1 of Cilofexor:Tromethamine may be added to the reaction mixture, preferably at a temperature of: about 80° C. to about 45° C., or about 70° C. to about 50° C., or about 50° C. to about 55° C., preferably in an amount of: about 25 mg to about 70 mg, about 30 mg to about 65 mg, or about 50 mg, of seeds of form CT1 of Cilofexor:Tromethamine per gram of Cilofexor.

The process may further include isolating the obtained Cilofexor: Tromethamine [step (v)], by any suitable procedure, such as filtration, decantation, or by centrifuge. Particularly the product may be isolated by vacuum filtration. Following isolation, the Cilofexor:Tromethamine is dried [step (vi)]. The drying may be carried out, for example, during filtration for the period of: about 5 minutes to 1 hour, or about 10 minutes to about 15 minutes. Optionally, and preferably additionally, the Cilofexor: Tromethamine is dried in a Vacuum tray dryer (VTD), typically at a temperature of: about 40° C. to about 90° C., about 30° C. to about 85° C., or about 80° C. The drying may be carried out for any suitable time to remove the solvent (e.g. to a constant weight), typically for: about 10 hours to about 30 hours, about 12 hours to about 24 hours, about 16 hours to about 20 hours, or about 18 hours to about 20 hours.

According to any aspect or embodiment, the processes for preparing Form CT1 of Cilofexor:Tromethamine may further comprise combining the Form CT1 of Cilofexor:Tromethamine with at least one pharmaceutically acceptable excipient to prepare a pharmaceutical composition or formulation.

The present disclosure includes a crystalline polymorph of Cilofexor: Tromethamine, designated Form CT2. The crystalline Form CT2 of Cilofexor: Tromethamine may be characterized by data selected from one or more of the following: an X-ray powder diffraction pattern substantially as depicted in FIG. 2; an X-ray powder diffraction pattern having peaks at 8.6, 15.3, 17.1, 19.4 and 22.1 degrees 2-theta±0.2 degrees 2-theta; and combinations of these data.

Crystalline Form CT2 of Cilofexor:Tromethamine may be further characterized by an X-ray powder diffraction pattern having peaks at 8.6, 15.3, 17.1, 19.4 and 22.1 degrees 2-theta±0.2 degrees 2-theta, and also having any one, two, three, four or five additional peaks selected from 7.4, 16.1, 18.5, 23.7 and 29.9 degrees 2-theta±0.2 degrees 2-theta.

In any aspect or embodiment of the disclosure, crystalline Form CT2 of Cilofexor:Tromethamine may be characterized by any of the above data, and additionally having an absence of peaks at 3.0-3.9 degrees 2-theta±0.2 degrees 2-theta; and/or an absence of peaks at 4.9-6.9 degrees 2-theta±0.2 degrees 2-theta; and/or an absence of peaks at 9.4-9.9 degrees 2-theta±0.2 degrees 2-theta. Alternatively, in any aspect or embodiment of the disclosure, crystalline Form CT2 of Cilofexor:Tromethamine may be characterized by any of the above data, and additionally having an absence of peaks at 4.9-6.9 degrees 2-theta±0.2 degrees 2-theta; and/or an absence of peaks at 9.4-9.9 degrees 2-theta±0.2 degrees 2-theta.

In one embodiment of the present disclosure, crystalline Form CT2 of Cilofexor:Tromethamine is isolated. Particularly, according to any aspect or embodiment, crystalline Form CT2 of Cilofexor:Tromethamine is isolated.

In one embodiment of the present disclosure, crystalline Form CT2 of Cilofexor:Tromethamine may be THF solvate. Particularly, according to any aspect or embodiment, crystalline Form CT2 of Cilofexor:Tromethamine is a THF solvate.

Crystalline Form CT2 of Cilofexor:Tromethamine may be characterized by an X-ray powder diffraction pattern having peaks at 7.4, 8.6, 15.3, 16.1, 17.1, 18.5, 19.4, 22.1, 23.7 and 29.9 degrees 2-theta±0.2 degrees 2-theta.

According to any aspect or embodiment of the disclosure, the crystalline Form CT2 of Cilofexor:Tromethamine may be polymorphically pure. The crystalline Form CT2 of Cilofexor:Tromethamine according to any aspect or embodiment of the disclosure, may be chemically pure.

Crystalline Form CT2 of Cilofexor:Tromethamine may be characterized by each of the above characteristics alone/or by all possible combinations, e.g., an XRPD pattern having peaks at 8.6, 15.3, 17.1, 19.4 and 22.1 degrees 2-theta±0.2 degrees 2-theta; an XRPD pattern as depicted in FIG. 2, and combinations thereof.

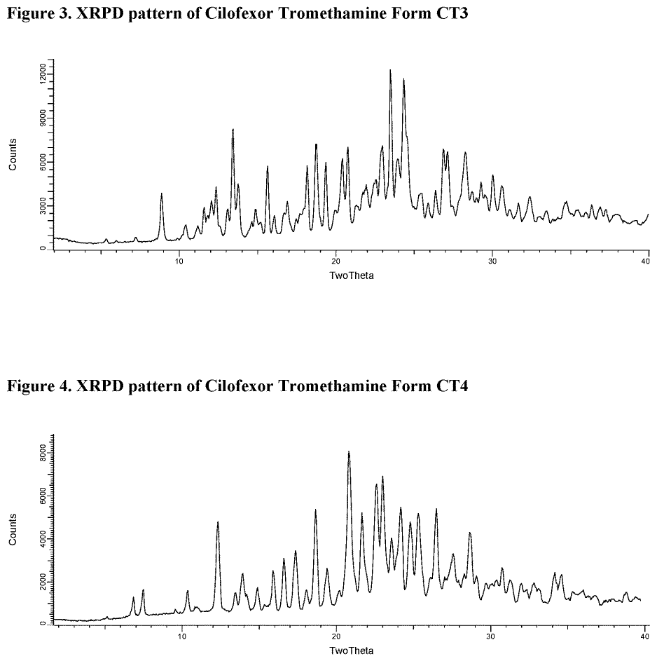

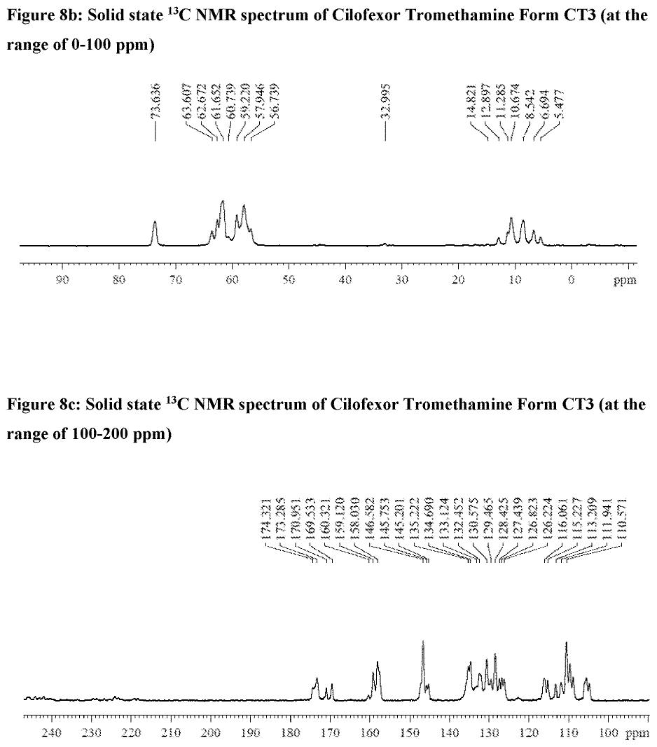

The present disclosure includes a crystalline polymorph of Cilofexor: Tromethamine, designated Form CT3. The crystalline Form CT3 of Cilofexor: Tromethamine may be characterized by data selected from one or more of the following: an X-ray powder diffraction pattern substantially as depicted in FIG. 3; an X-ray powder diffraction pattern having peaks at 8.9, 12.3, 15.6, 18.7 and 20.7 degrees 2-theta±0.2 degrees 2-theta; a solid state 13C NMR spectrum having peaks at 11.3, 104.8, 116.1, 132.5, 134.7 and 169.5 ppm ppm±0.2 ppm; a solid state 13C NMR spectrum having the following chemical shift absolute differences from a reference peak at 173.3 ppm±2 ppm of 162.0, 68.5, 57.2, 40.8, 38.6 and 3.8±0.1 ppm; a solid state 13C NMR spectrum substantially as depicted in FIGS. 8a, 8b or 8c; and combinations of these data.

Crystalline Form CT3 of Cilofexor:Tromethamine may be further characterized by an X-ray powder diffraction pattern having peaks at 8.9, 12.3, 15.6, 18.7 and 20.7 degrees 2-theta±0.2 degrees 2-theta, and also having any one, two, three, or four additional peaks selected from 19.4, 23.5, 28.3 and 30.0 degrees 2-theta±0.2 degrees 2-theta.

In any aspect or embodiment of the disclosure, crystalline Form CT3 of Cilofexor:Tromethamine may be characterized by any of the above data, and additionally having an absence of peaks at 3.0-4.8 degrees 2-theta±0.2 degrees 2-theta; and/or an absence of peaks at 7.7-8.4 degrees 2-theta±0.2 degrees 2-theta.

The molar ratio between Cilofexor and Tromethamine may be 1:1. Thus, according to any aspect or embodiment of the disclosure, the molar ratio of Cilofexor and Tromethamine in crystalline Form CT3 of Cilofexor:Tromethamine is 1:1.

In one embodiment of the present disclosure, crystalline Form CT3 of Cilofexor:Tromethamine is isolated. Particularly, according to any aspect or embodiment, crystalline Form CT3 of Cilofexor:Tromethamine is isolated.

In one embodiment of the present disclosure, crystalline Form CT3 of Cilofexor:Tromethamine may be an anhydrous form. Particularly, according to any aspect or embodiment, crystalline Form CT3 of Cilofexor:Tromethamine is an anhydrous form.

Crystalline Form CT3 of Cilofexor:Tromethamine may be characterized by an X-ray powder diffraction pattern having peaks at 8.9, 12.3, 15.6, 18.7, 19.4, 20.7, 23.5, 28.3 and 30.0 degrees 2-theta±0.2 degrees 2-theta.

According to any aspect or embodiment of the disclosure, the crystalline Form CT3 of Cilofexor:Tromethamine may be polymorphically pure. The crystalline Form CT3 of Cilofexor:Tromethamine according to any aspect or embodiment of the disclosure, may be chemically pure.

Crystalline Form CT3 of Cilofexor:Tromethamine may be characterized by each of the above characteristics alone/or by all possible combinations, e.g., an XRPD pattern having peaks at 8.9, 12.3, 15.6, 18.7 and 20.7 degrees 2-theta±0.2 degrees 2-theta; an XRPD pattern as depicted in FIG. 3, and combinations thereof.

The present disclosure further provides a process for preparing crystalline Form CT3 of Cilofexor:Tromethamine, as described in any aspect or embodiment of the disclosure.

The process may comprise crystallising Cilofexor:Tromethamine from either propionitrile or a mixture of THF, propionitrile and n-heptane. Particularly, the process may comprise:
(a) preparing a mixture of Cilofexor:Tromethamine in either propionitrile or a mixture of THF, propionitrile and n-heptane;
(b) crystallising the Cilofexor:Tromethamine from the mixture;
(c) optionally isolating the crystals; and
(d) optionally drying.
   According to any aspect or embodiment of the process, the solution in step (a) may be prepared by combining Cilofexor, Tromethamine, and either propionitrile or a mixture of THF, propionitrile and n-heptane, to form a mixture, and preferably heating the mixture. Alternatively, the solution in step (a) may be prepared by combining Cilofexor with propionitrile, or a mixture of THF, propionitrile and n-heptane, and combining with Tris(hydroxymethyl)aminomethane. In an alternative embodiment, the solution in step (a) may be prepared by combining Cilofexor in propionitrile, or a mixture of THF, propionitrile and n-heptane, with a solution of Tris(hydroxymethyl)aminomethane in THF or propionitrile. Preferably, according to any aspect or embodiment of the process, the mixture in step (a) is prepared by combining Cilofexor:Tromethamine and either propionitrile or a mixture of THF, propionitrile and n-heptane. Further alternatively, the solution in step (a) may be prepared by combining Cilofexor:Tromethamine with THF, and combining the solution with a mixture of propionitrile and n-heptane. Preferably, when the mixture in step (a) is a mixture of Cilofexor:Tromethamine in propionitrile, the Cilofexor:Tromethamine is Form CT2; typically the mixture is a slurry. According to any aspect or embodiment of the process, the combining of the Cilofexor or Cilofexor Tromethamine with the solvent, may be carried out at a temperature of: about 15° C. to about 30° C., about 18° C. to about 28° C., about 20° C. to about 25° C., particularly about 25° C. According to any aspect or embodiment of the process, the mixture in step (a) may be heated to a temperature of: about 40° C. to about 80° C., about 40° C. to about 70° C., about 45° C. to about 65° C., or about 50° C. to about 60° C., or about 50° C., or about 50° C. to about 55° C. Alternatively, in any embodiment of the process, where the mixture of Cilofexor: Tromethamine in step (a) is in THF, propionitrile and n-heptane is used, preferably Cilofexor:Tromethamine is first dissolved in THF at a temperature of: about 40° C. to about 80° C., about 40° C. to about 70° C., about 45° C. to about 65° C., or about 50° C. to about 60° C., or about 50° C., or about 50° C. to about 55° C., and combined with a mixture of propionitrile and n-heptane; preferably by adding the solution of Cilofexor: Tromethamine salt in THF to the mixture of propionitrile and n-heptane. Preferably, the solution of Cilofexor:Tromethamine is heated to a temperature of: about 40° C. to about 80° C., about 40° C. to about 70° C., about 45° C. to about 65° C., or about 50° C. to about 60° C., or about 50° C., or about 50° C. to about 55° C. and optionally hot-filtered prior to combining with a mixture of propionitrile. The mixture of propionitrile and n-heptane may contain seeds of Form CT3. Preferably, the seeds are used in an amount of: about 25 mg to about 60 mg, about 30 mg to about 50 mg, or about 40 mg, per gram of Cilofexor: Tromethamine. According to any aspect or embodiment of the process wherein the mixture in step (a) is a mixture of Cilofexor:Tromethamine in propionitrile, the propionitrile may be used in an amount of: about 10 ml to about 40 ml per gram of Cilofexor:Tromethamine, about 12 ml to about 30 ml per gram of Cilofexor: Tromethamine, about 15 ml to about 25 ml per gram of Cilofexor:Tromethamine, about 18 ml to about 22 ml per gram of Cilofexor:Tromethamine, or about 20 ml per gram of Cilofexor:Tromethamine. In any embodiment of the process, where the mixture of Cilofexor:Tromethamine in step (a) is in THF, propionitrile and n-heptane, the propionitrile may be used in an amount of: about 4 ml to about 20 ml per gram of Cilofexor:Tromethamine, about 5 ml to about 18 ml per gram of Cilofexor: Tromethamine, about 6 ml to about 15 ml per gram of Cilofexor:Tromethamine, about 8 ml to about 12 ml per gram of Cilofexor:Tromethamine, or about 10 ml per gram of Cilofexor:Tromethamine. In any embodiment of the process, where the mixture of Cilofexor:Tromethamine in step (a) is in THF, propionitrile and n-heptane is used, the THE may be used in an amount of: about 0.5 ml to about 10 ml per gram of Cilofexor: Tromethamine, about 0.8 ml to about 5 ml per gram of Cilofexor:Tromethamine, about 1.0 ml to about 2 ml per gram of Cilofexor:Tromethamine, about 1.2 ml to about 1.8 ml per gram of Cilofexor:Tromethamine, or about 1.5 ml per gram of Cilofexor: Tromethamine. In any embodiment of the process, where the mixture of Cilofexor: Tromethamine in step (a) is in THF, propionitrile and n-heptane is used, the n-heptane may be used in an amount of: about 10 ml to about 40 ml per gram of Cilofexor: Tromethamine, about 12 ml to about 35 ml per gram of Cilofexor:Tromethamine, about 15 ml to about 30 ml per gram of Cilofexor:Tromethamine, about 18 ml to about 25 ml per gram of Cilofexor:Tromethamine, or about 22 ml per gram of Cilofexor: Tromethamine. In embodiments where the solution of Cilofexor:Tromethamine in step (a) is in THF, propionitrile and n-heptane, the vol:vol ratio of THF to propionitrile may be in a range of: about 1:1 to about 1:20, about 1:4 to about 1:15, about 1:5 to about 1:10, or about 1:6; and/or the vol:vol ratio of THF to n-heptane may be in the range of: about 1:5 to about 1:30, about 1:10 to about 1:20, about 1:12 to about 1:16, or about 1:14. In any aspect or embodiment of the process for preparing Form CT3 of Cilofexor: Tromethamine, the molar ratio of Cilofexor and Tromethamine is about 1:1. In any aspect or embodiment of the process wherein the mixture in step (a) is a mixture of Cilofexor: Tromethamine in propionitrile, step (b) may comprise stirring the mixture at a temperature of: about 40° C. to about 70° C., about 45° C. to about 65° C., about 45° C. to about 60° C., or about 50° C. to about 55° C., preferably for a period of: about 6 hours to about 60 hours, about 10 hours to about 36 hours, about 15 hours to about 24 hours, about 16 hours to about 20 hours, or about 18 hours. In any embodiment of the process wherein the mixture in step (a) is a mixture of Cilofexor:Tromethamine in a mixture of THF, propionitrile and n-heptane, step (b) may comprise stirring the mixture at a temperature of: about 35° C. to about 60° C., about 40° C. to about 50° C., about 40° C. to about 55° C., or about 45° C., preferably for a period of: about 6 hours to about 60 hours, about 10 hours to about 36 hours, about 15 hours to about 24 hours, about 16 hours to about 20 hours, or about 18 hours. According to any aspect or embodiment of the process, the resulting crystals may be isolated in step (c) by any suitable method, such as by filtration, decantation or centrifugation, preferably by filtration. According to any aspect or embodiment of the process, the crystals may be dried in step (d) to remove the solvent, for example to a constant weight. Optionally step (d) may comprise drying during suction filtration for the period of about 5 minutes to 2 hours, or about 10 minutes to about 1.5 hour, or about 15 minutes to about 1 hour. According to any aspect or embodiment of the process, step (d) optionally or preferably additionally comprises drying the crystals at a temperature of: about 60° C. to about 100° C., about 65° C. to about 95° C., about 60° C. to about 80° C., or about 70° C. to about 90° C., about 75° C. to about 85° C., or about 80° C., preferably under reduced pressure or under vacuum. Preferably the drying can be carried out for a suitable period of time to remove the solvent, more preferably to a constant weight. According to any aspect or embodiment of the process, the drying may be carried out at a temperature of: about 70° C. to about 90° C., about 75° C. to about 85° C., or about 60° C. to about 80° C., or about 80° C., under reduced pressure or vacuum (more particularly in a vacuum tray dryer), preferably for about 8 hours to about 48 hours, about 10 hours to about 30 hours, about 12 hours to about 24 hours, about 16 hours to about 20 hours, or about 18 hours to about 20 hours.

According to any aspect or embodiment of the disclosed processes for preparing form CT3 of Cilofexor:Tromethamine, the process may comprise:
(i) combining Form CT2 of Cilofexor:Tromethamine with propionitrile;
(ii) heating the mixture, optionally with stirring;
(iii) isolating the Cilofexor:Tromethamine; and optionally
(iv) drying the Cilofexor:Tromethamine to form Cilofexor:Tromethamine Form CT3.

According to any aspect or embodiment of the disclosed processes, in step (i), propionitrile may be added to Form CT2 of Cilofexor:Tromethamine. Particularly, propionitrile may be added at an amount of about 5 to about 20 ml, or about 16 ml of propionitrile per gram of Form CT2 of Cilofexor:Tromethamine. Propionitrile may be added step (i) at temperature of: about 55° C. to about 40° C., or about 50° C. to about 35° C., or about 25° C.

In any aspect or embodiment of the disclosed processes, step (ii), reaction mixture may be heated to temperature of: about 80° C. to about 45° C., or about 70° C. to about 50° C., or about 50° C. to about 55° C.

In any aspect or embodiment of the disclosed processes, step (ii), the solution may be stirred at temperature of about temperature of: about 80° C. to about 45° C., or about 70° C. to about 50° C., or about 50° C. to about 55° C. for the period of: about 30 hours to about 25 hours, or about 25 hours to about 20 hours, or about 18 hours.

The process may further include isolating the obtained Cilofexor: Tromethamine Form CT3 [step (iii)], by any suitable procedure, such as filtration, decantation, or by centrifuge. Particularly the product may be isolated by vacuum filtration. Following isolation, the Cilofexor:Tromethamine Form CT3 may be dried. The drying [step (iv)] may be carried out during, e.g. filtration for the period of: about 20 minutes to 40 hour, or about 15 minutes to about 30 minutes at temperature of: about 40° C. to about 60° C., about 30° C. to about 50° C., or about 25° C. Optionally or preferably additionally, the Cilofexor:Tromethamine Form CT3 may be dried in a Vacuum tray dryer (VTD), typically at a temperature of: about 40° C. to about 90° C., about 30° C. to about 85° C., or about 80° C. The drying may be carried out for any suitable time to remove the solvent (e.g. to a constant weight), typically: about 15 hours to about 25 hours, or about 20 hours.

According to any aspect or embodiment of the disclosed processes for preparing Form CT3 of Cilofexor:Tromethamine, the process may comprise:
(i) dissolving Cilofexor Tromethamine salt in tetrahydrofuran;
(ii) providing a mixture comprising seeds of form CT3 in a mixture of propionitrile: n-heptane mixture;
(iii) adding solution of step (i) to the mixture in step (ii), and optionally stirring the mixture;
(iv) isolating of the Cilofexor:Tromethamine; and optionally
(v) drying the Cilofexor:Tromethamine to form Cilofexor:Tromethamine form CT3.

According to any aspect or embodiment of the disclosed processes, in step (i), Cilofexor Tromethamine salt may be dissolved in tetrahydrofuran at temperature of: about 80° C. to about 45° C., or about 70° C. to about 50° C., or about 50° C. to about 55° C.

Particularly, the tetrahydrofuran in step (i) may be added at an amount of: about 4 to about 1 ml, or about 1.6 ml of tetrahydrofuran per gram of Cilofexor Tromethamine salt.

Seeds of form CT3 in step (ii) may be added to solvent mixture of propionitrile: n-heptane at an amount of: about 20 mg to about 60 mg, or about 40 mg of seeds of form CT3 per gram of Cilofexor Tromethamine salt. Propionitrile: n-heptane may be added at ratio of: about 10% to about 40%, or about 30% Propionitrile: n-heptane may be added at an amount of about 20 ml to about 60 ml, or about 32 ml of propionitrile: n-Heptane per gram of Cilofexor tromethamine salt.

In any aspect or embodiment of the disclosed processes, step (iii), solution of step (i) may be added to mixture of step (ii) at temperature of: about 80° C. to about 45° C., or about 70° C. to about 50° C., or about 50° C. to about 55° C. In any aspect or embodiment of the disclosed processes, in step (iii), the mixture may be stirred at temperature of about temperature of: about 80° C. to about 45° C., or about 70° C. to about 50° C., or about 45° C. for the period of: about 10 hours to about 30 hours, about 12 hours to about 24 hours, about 16 hours to about 20 hours, or about 18 hours.

The process may further include isolating the obtained Cilofexor: Tromethamine Form CT3, by any suitable procedure, such as filtration, decantation, or by centrifuge. Particularly the product may be isolated by vacuum filtration. Following isolation, the Cilofexor:Tromethamine Form CT3 may be dried. The drying may be carried out during, e.g. filtration for the period of about 5 minutes to 2 hours, or about 30 minutes to about 1.5 hour, or about 1 hour. Optionally or preferably additionally, the Cilofexor:Tromethamine Form CT3 may be dried in a Vacuum tray dryer (VTD), typically at a temperature of about 40° C. to about 90° C., about 30° C. to about 85° C., or about 60° C. to about 80° C. The drying may be carried out for any suitable time to remove the solvent (e.g. to a constant weight), typically: about 10 hours to about 30 hours, about 12 hours to about 24 hours, about 16 hours to about 20 hours, or about 18 hours.

According to any aspect or embodiment, the processes for preparing Form CT3 of Cilofexor:Tromethamine may further comprise combining the Form CT3 of Cilofexor:Tromethamine with at least one pharmaceutically acceptable excipient to prepare a pharmaceutical composition or formulation.

The present disclosure includes a crystalline polymorph of Cilofexor: Tromethamine, designated Form CT4. The crystalline Form CT4 of Cilofexor: Tromethamine may be characterized by data selected from one or more of the following: an X-ray powder diffraction pattern substantially as depicted in FIG. 4; an X-ray powder diffraction pattern having peaks at 10.4, 12.3, 18.7, 20.8 and 26.5 degrees 2-theta±0.2 degrees 2-theta; and combinations of these data.

Crystalline Form CT4 of Cilofexor:Tromethamine may be further characterized by an X-ray powder diffraction pattern having peaks at 10.4, 12.3, 18.7, 20.8 and 26.5 degrees 2-theta±0.2 degrees 2-theta, and also having any one, two, three, four, or five additional peaks selected from 6.9, 15.9, 19.4, 21.7 and 27.6 degrees 2-theta±0.2 degrees 2-theta.

In one embodiment of the present disclosure, crystalline Form CT4 of Cilofexor:Tromethamine is isolated. Particularly, according to any aspect or embodiment, crystalline Form CT4 of Cilofexor:Tromethamine is isolated.

In one embodiment of the present disclosure, crystalline Form CT4 of Cilofexor:Tromethamine may be 2-butoxyethanol solvate. Particularly, according to any aspect or embodiment, crystalline Form CT4 of Cilofexor:Tromethamine is a 2-butoxyethanol solvate.

Crystalline Form CT4 of Cilofexor:Tromethamine may be characterized by an X-ray powder diffraction pattern having peaks at 6.9, 10.4, 12.3, 15.9, 18.7, 19.4, 20.8, 21.7, 26.5 and 27.6 degrees 2-theta±0.2 degrees 2-theta.

According to any aspect or embodiment of the disclosure, the crystalline Form CT4 of Cilofexor:Tromethamine may be polymorphically pure. Particularly, according to any aspect or embodiment of the disclosure, crystalline Form CT4 of Cilofexor: Tromethamine is polymorphically pure. The crystalline Form CT4 of Cilofexor: Tromethamine according to any aspect or embodiment of the disclosure, may be chemically pure.

Crystalline Form CT4 of Cilofexor:Tromethamine may be characterized by each of the above characteristics alone/or by all possible combinations, e.g., an XRPD pattern having peaks at 6.9, 15.9, 19.4, 21.7 and 27.6 degrees 2-theta±0.2 degrees 2-theta; an XRPD pattern as depicted in FIG. 4, and combinations thereof.

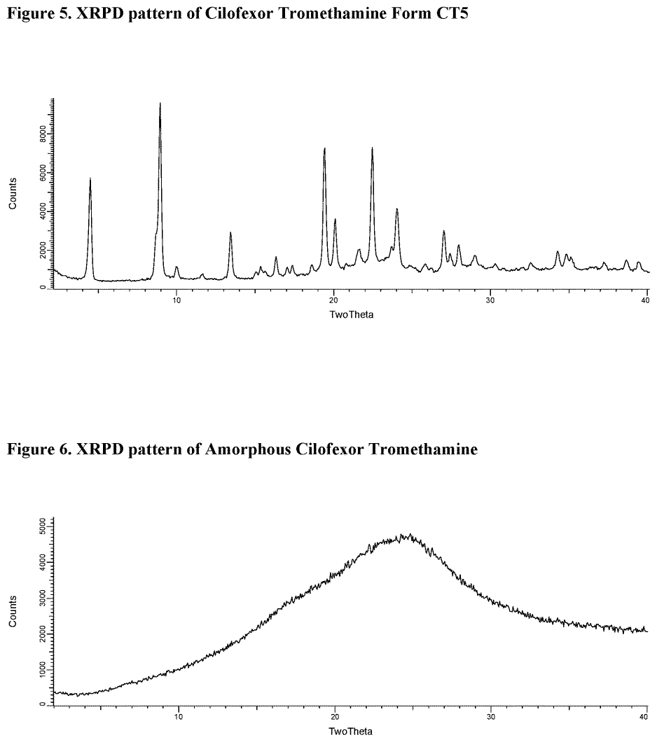

The present disclosure includes a crystalline polymorph of Cilofexor: Tromethamine, designated Form CT5. The crystalline Form CT5 of Cilofexor: Tromethamine may be characterized by data selected from one or more of the following: an X-ray powder diffraction pattern substantially as depicted in FIG. 5; an X-ray powder diffraction pattern having peaks at 4.5, 8.9, 13.4, 22.5 and 27.0 degrees 2-theta±0.2 degrees 2-theta; and combinations of these data.

Crystalline Form CT5 of Cilofexor:Tromethamine may be further characterized by an X-ray powder diffraction pattern having peaks at 4.5, 8.9, 13.4, 22.5 and 27.0 degrees 2-theta±0.2 degrees 2-theta, and also having any one, two, three, four, or five additional peaks selected from 16.3, 19.4, 20.1, 24.0 and 34.3 degrees 2-theta 0.2 degrees 2-theta.

In one embodiment of the present disclosure, crystalline Form CT5 of Cilofexor:Tromethamine is isolated. Particularly, according to any aspect or embodiment, crystalline Form CT5 of Cilofexor:Tromethamine is isolated.

In one embodiment of the present disclosure, crystalline Form CT5 of Cilofexor:Tromethamine may be 1-Methoxy-2-propanol Solvate. Particularly, according to any aspect or embodiment, crystalline Form CT5 of Cilofexor:Tromethamine is a 1-methoxy-2-propanol solvate.

Crystalline Form CT5 of Cilofexor:Tromethamine may be characterized by an X-ray powder diffraction pattern having peaks at 4.5, 8.9, 13.4, 16.3, 19.4, 20.1, 24.0, 22.5, 27.0 and 34.3 degrees 2-theta±0.2 degrees 2-theta.

According to any aspect or embodiment of the disclosure, the crystalline Form CT5 of Cilofexor:Tromethamine may be polymorphically pure. The crystalline Form CT5 of Cilofexor:Tromethamine according to any aspect or embodiment of the disclosure, may be chemically pure.

Crystalline Form CT5 of Cilofexor:Tromethamine may be characterized by each of the above characteristics alone/or by all possible combinations, e.g., an XRPD pattern having peaks at 4.5, 8.9, 13.4, 22.5 and 27.0 degrees 2-theta±0.2 degrees 2-theta; an XRPD pattern as depicted in FIG. 5, and combinations thereof.

The above crystalline forms can be used to prepare other crystalline forms of Cilofexor, Cilofexor salts and their solid state forms.

The present disclosure encompasses a process for preparing other solid state forms of Cilofexor, Cilofexor salts and their solid state forms thereof. The process includes preparing any one of the Cilofexor (salts) and solid state forms thereof as disclosed herein by the processes of the present disclosure, and converting it to other Cilofexor salt(s) and solid state forms thereof.

The present disclosure provides the above described solid state forms of Cilofexor for use in the preparation of pharmaceutical compositions comprising Cilofexor and salts thereof and/or solid state forms thereof. In particular the present disclosure encompasses the use of the above described solid state forms of Cilofexor and salts thereof, for the preparation of a pharmaceutical composition in the form of a tablet comprising Cilofexor or salt thereof.

The present disclosure also encompasses the use of solid state forms of Cilofexor of the present disclosure for the preparation of pharmaceutical compositions of Cilofexor and salts thereof and/or solid state forms thereof. In particular the present disclosure encompasses the above described solid state forms of Cilofexor and salts thereof, for the preparation of a pharmaceutical composition or formulation, preferably an oral formulation in the form of a tablet comprising Cilofexor or salt thereof.

The present disclosure includes processes for preparing the above mentioned pharmaceutical compositions. The processes include combining any one or a combination of the solid state forms of Cilofexor of the present disclosure with at least one pharmaceutically acceptable excipient.

Pharmaceutical combinations or formulations of the present disclosure contain any one or a combination of the solid state forms of Cilofexor of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g., Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g., carbopol), carboxymethylcellulose sodium, dextrin, ethyl cellulose, gelatin, guar gum, hydrogenated vegetable oil, hydroxyethyl cellulose, hydroxypropyl cellulose (e.g., Klucel®), hydroxypropyl methyl cellulose (e.g., Methocel®), liquid glucose, magnesium aluminum silicate, maltodextrin, methylcellulose, polymethacrylates, povidone (e.g., Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g., Ac-Di-Sol®, Primellose®), colloidal silicon dioxide, croscarmellose sodium, crospovidone (e.g., Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g., Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Cilofexor and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Cilofexor can be administered. Cilofexor may be formulated for administration to a mammal, in embodiments to a human, by injection. Cilofexor can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Cilofexor and the pharmaceutical compositions and/or formulations of Cilofexor of the present disclosure can be used as medicaments, in embodiments in the treatment of liver disease, Primary sclerosing cholangitis, Non-alcoholic fatty liver disease; Non-alcoholic steatohepatitis and Primary biliary cirrhosis.

The present disclosure also provides methods of treating liver disease, Primary sclerosing cholangitis, Non-alcoholic fatty liver disease; Non-alcoholic steatohepatitis and Primary biliary cirrhosis by administering a therapeutically effective amount of any one or a combination of the crystalline polymorphs of Cilofexor of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

Solid State ¹³C-NMR (¹³C CP/MAS NMR) Method

Solid-state NMR spectra were measured at 11.7 T using a Bruker Avance III HD 500 US/WB NMR spectrometer (Karlsruhe, Germany, 2013) with 3.2 mm probehead. The 13C CP/MAS NMR spectra employing cross-polarization were acquired using the standard pulse scheme at spinning frequency of 18 kHz and a room temperature (300 K). The recycle delay was 8 s and the cross-polarization contact time was 2 ms. The 13C scale was referenced to α-glycine (176.03 ppm for ¹³C). Frictional heating of the spinning samples was offset by active cooling, and the temperature calibration was performed with Pb(NO3)2. The NMR spectrometer was completely calibrated and all experimental parameters were carefully optimized prior the investigation. Magic angle was set using KBr during standard optimization procedure and homogeneity of magnetic field was optimized using adamantane sample (resulting line-width at half-height Δυ1/2 was less than 3.5 Hz at 250 ms of acquisition time).

### Powder X-Ray Diffraction (“XRPD”) Method

X-ray diffraction was performed on X-Ray powder diffractometer: Bruker D8 Advance; CuK_ radiation (λ=1.54 Å); Lynx eye detector; laboratory temperature 22-25° C.; PMMA specimen holder ring. Prior to analysis, the samples were gently ground by means of mortar and pestle in order to obtain a fine powder. The ground sample was adjusted into a cavity of the sample holder and the surface of the sample was smoothed by means of a cover glass.

### Measurement Parameters:

Scan range: 2-40 degrees 2-theta
Scan mode: continuous
Step size: 0.05 degrees
Time per step: 0.5 s
Sample spin: 30 rpm
Sample holder: PMMA specimen holder ring
   All X-Ray Powder Diffraction peak values are calibrated with regard to standard silicon spiking in the sample.

### EXAMPLES

### Preparation of Starting Materials

Cilofexor can be prepared according to methods known from the literature, for example U.S. Pat. No. 9,139,539. Cilofexor Tromethamine salt can be prepared according to methods known from the literature, for example, WO 2020/172075.

### Example 1: Preparation of Cilofexor:Tromethamine Form CT1

Cilofexor (0.3 grams) and Tris(hydroxyl-methyl)amino-methane (TRIS, 0.068 g) were charged in 10 ml reaction vial and tetrahydrofuran (THF, 4.5 ml) was added at 25° C. The reaction mixture was heated up to 60° C. The hot solution was filtered and clear solution maintained under stirring at 60° C. for about 48 hours. After 2 days, solid was crystallized out, filtered and suck dried for about 15-30 minutes. The sample was further dried in Vacuum tray dryer at 80° C. for about 16 hours. The obtained solid was analyzed by XRPD and designated as Cilofexor:Tromethamine Form CT1.

### Example 2: Preparation of Cilofexor:Tromethamine Form CT2

Cilofexor free base (0.3 grams) and Tris(hydroxyl-methyl)amino-methane (TRIS, 0.068 grams) was charged in 50 ml reaction flask and tetrahydrofuran (THF, 4.5 ml) was added at temperature of about 25° C. The reaction mixture was heated up to temperature of about 60° C. to about 65° C. to get clear solution. The hot clear solution was maintained under stirring at temperature of about 60° C. for period of about 4 hours and white solid formation was observed. The reaction mixture was maintained under stirring for period of about 55° C. to about 60° C. for period of about 16 hours. The obtained solid was filtered and suck dried for period of about 15 minutes to 30 minutes at temperature of about 25° C. The obtained solid was further dried in a Vacuum tray dryer (VTD) at temperature of about 30° C. for period of about 3 hours. The obtained solid was analyzed by XRPD. Cilofexor:Tromethamine Form CT2 was obtained. An XRPD pattern is shown in FIG. 2.

### Example 3: Preparation of Cilofexor:Tromethamine Form CT3

Cilofexor:Tromethamine (Form CT2) (1.0 gram) was charged in 50 ml reaction flask and Propionitrile (20 ml) was added at temperature of about 25° C. The reaction mixture was heated up to temperature of about 50° C. to about 55° C. The slurry mass was maintained under stirring for period of about 18 hours at a temperature of about 50° C. to about 55° C. The slurry mass was filtered and suck dried for period of about 15 minutes to about 30 minutes at temperature of about 25° C. The obtained solid was further dried in a Vacuum tray dryer (VTD) at temperature of about 80° C. for about 20 hours. The obtained solid was analyzed by XRPD. Cilofexor:Tromethamine Form CT3 was obtained. An XRPD pattern is shown in FIG. 3.

### Example 4: Preparation of Cilofexor:Tromethamine Form CT4

Cilofexor:Tromethamine (Amorphous) (0.02 grams) was charged in 10 ml reaction flask and 2-Butoxyethanol (0.1 ml) was added at temperature of about 25° C. The reaction mixture was heated up to temperature of about 50° C. to about 55° C. to get clear solution. The clear solution was slowly cooled to room temperature. The reaction mass maintained for period of about 6 hours at temperature of about 25° C. The white solid was filtered and suck dried for period of about 15 minutes to about 30 minutes at temperature of about 25° C. The obtained solid was analyzed by XRPD. Cilofexor:Tromethamine Form CT4 was obtained. An XRPD pattern is shown in FIG. 4.

### Example 5: Preparation of Cilofexor:Tromethamine Form CT5

Cilofexor Tromethamine salt (0.02 grams) was charged in 5 ml reaction flask and 1-Methoxy-2-propanol (0.1 ml) was added at temperature of about 25° C. The reaction mixture was heated up to temperature of about 50° C. to about 55° C. to get clear solution. The clear solution was added to the Chilled MIBK (2 ml). The reaction mixture was maintained for period of about 5 hours at a temperature of about 0° C. to about 5° C. The white solid mass was filtered and suck dry for period of about 10 minutes to about 15 minutes. The obtained solid was analyzed by XRPD. Cilofexor:Tromethamine Form CT5 was obtained. An XRPD pattern is shown in FIG. 5.

### Example 6: Preparation of Amorphous Cilofexor:Tromethamine

Cilofexor Tromethamine salt (1.0 g) was dissolved in acetone (30 ml) at temperature of about 40° C. The solution was filtered and clear solution was added into precooled diisopropyl ether (150 ml) at temperature of about −10° C. The reaction mixture was maintained under stirring for period of about 6 hours at temperature of about −10° C. and the milky solution was observed. The temperature of reaction mass was increased to temperature of about 0° C. to about 5° C. and maintained under stirring for period of about 30 minutes. The obtained solid was filtered and washed with diisopropyl ether (10 ml) and suck dried for period of about 10 minutes to about 15 minutes. The obtained solid was analyzed by XRPD. Amorphous Cilofexor:Tromethamine was obtained. An XRPD pattern is shown in FIG. 6.

### Example 7: Preparation of Cilofexor:Tromethamine Form CT1

Cilofexor (1.0 gram) charged in 100 ml four neck RBF and added tetrahydrofuran (THF, 15.0 mL) at 25° C. The reaction mixture was heated up to 50-55° C. and stirred at 300 RPM for about 15 min and obtained a suspension. In another vial, Tris(hydroxyl-methyl)amino-methane (TRIS, 0.226 grams) was dissolved in water (0.34 mL; 1.5V) at about 55° C. The clear TRIS solution (0.34 ml) was added in to the above suspension which resulted into a clear solution at about 50-55° C. Then added seeds of Form CT1 (about 50 mg) into the reaction mass at about 50-55° C. The reaction mixture was maintained under stirring at about 50-55° C. for about 8 hours. After 8 h, the reaction mass was filtered and suck dried for about 10-15 minutes. The sample was further dried in Vacuum tray dryer at about 80° C. for about 18 hours. The obtained solid was analyzed by XRPD. Form CT1 of Cilofexor Tromethamine salt was obtained.

### Example 8: Preparation of Cilofexor:Tromethamine Form CT3

Cilofexor Tromethamine salt (5.0 grams) in a 10 mL conical flask and was dissolved with THF (8 mL) at 50-55° C. The clear solution was particle free through micron filtration. In another flask, 160 ml (30% Propionitrile: n-Heptane) was charged in to the 250 mL RBF and the reaction mixture was heated up to 50-55° C. and added seeds of Form CT3 (about 200 mg), the reaction mixture was stirred for 10 minutes at about 50-55° C. and the clear solution of Cilofexor Tromethamine salt was added into it. The reaction mass was maintained for 18 hours at about 45° C. The obtained white solid was filtered and suck dried for about 1 hour. The obtained solid was further dried in Vacuum tray dryer at 60-80° C. for about 18 hours. The obtained solid was analyzed by XRPD. Form CT3 of Cilofexor Tromethamine salt was obtained.

### Example 9: Stability Studies

### Storage Stability at Different Relative Humidities

Samples of Cilofexor:Tromethamine Forms CT1 and CT3 were subjected to conditions of different relative humidities at ambient temperature. XRPD analysis was performed on the samples after 7 days. The results are shown in Table 1 below:

TABLE 1

| | XRPD analysis results | | | |
|---|---|---|---|---|
| | Relative humidity/7 days | | | |
| Starting Form | 20% | 40% | 60% | 80% |
| Form CT1 | Form CT1 | Form CT1 | Form CT1 | Form CT1 |
| Form CT3 | Form CT3 | Form CT3 | Form CT3 | Form CT3 |

These results demonstrate that Forms CT1 and CT3 of Cilofexor:Tromethamine are stable after exposure to high and low relative humidity for at least 7 days.

### Thermal Stability

Samples of Cilofexor:Tromethamine Forms CT1 and CT3 were subjected to heating up to 80° C. and 100° C. for 30 minutes. XRPD analysis of the samples confirmed no polymorph change in the starting material (Table 2), confirming the high stability of Forms CT1 and CT3:

TABLE 2

XRPD analysis

| | Heating 80 °C, | Heating 100 °C, |
|---|---|---|
| Starting Form | 30 minutes | 30 minutes |
| Form CT1 | Form CT1 | Form CT1 |
| Form CT3 | Form CT3 | Form CT3 |

### Stability to Compression

Samples of Cilofexor:Tromethamine Forms CT1 and CT3 were subjected to pressures of 2 tons (Atlas® Autopress hydraulic press, set to 2 tons). XRPD analysis was performed on the samples after 1 minute. The results are shown in Table 3 below:

TABLE 3

| Starting Form | XRPD analysis results |
|---|---|
| Form CT1 | Form CT1 |
| Form CT3 | Form CT3 |

Accordingly, forms CT1 and CT3 of Cilofexor:Tromethamine are stable under high pressure conditions, making these forms highly suitable for pharmaceutical processing.

1. A crystalline Cilofexor:Tromethamine designated as Form CT1, which is characterized by data selected from:
a) an X-ray powder diffraction pattern having peaks at 5.7, 16.1, 20.2, 21.5 and 23.8 degrees 2-theta±0.2 degrees 2-theta;
b) a solid state ¹³C NMR spectrum having peaks at 57.6, 111.0, 129.1, 147.3, 158.8 and 165.8±0.2 ppm;
c) a solid state ¹³C NMR spectrum having the following chemical shift absolute differences from a reference peak at 173.0 ppm±2 ppm of 115.4, 62.0, 43.9, 25.7, 14.2 and 7.2 ppm±0.1 ppm;
d) a solid-state ¹³C NMR spectrum substantially as depicted in FIGS. 7a, 7b or 7c; and
e) combinations of two or more of: a, b, c, or d.

2. The crystalline Cilofexor:Tromethamine according to claim 1, which is characterized by an X-ray powder diffraction pattern having peaks at 5.7, 16.1, 20.2, 21.5 and 23.8 degrees 2-theta±0.2 degrees 2-theta, and also having any one, two, three, or four additional peaks selected from 10.8, 17.2, 18.2 and 34.1 degrees 2-theta±0.2 degrees 2-theta; or which is characterized by an XRPD pattern having peaks at: 5.7, 10.8, 16.1, 17.2, 18.2, 20.2, 21.5, 23.8 and 34.1 degrees 2-theta±0.2 degrees 2-theta; or an X-ray powder diffraction pattern substantially as depicted in FIG. 1.

3. The crystalline Cilofexor:Tromethamine according to claim 1, which is further characterized by an absence of peaks at 4.5-5.2 degrees 2-theta±0.2 degrees 2-theta; and/or an absence of peaks at 8.6-9.9 degrees 2-theta±0.2 degrees 2-theta.

4. A crystalline Cilofexor:Tromethamine designated as Form CT3, which is characterized by data selected from:
a) an X-ray powder diffraction pattern having peaks at 8.9, 12.3, 15.6, 18.7 and 20.7 degrees 2-theta±0.2 degrees 2-theta;
b) a solid state ¹³C NMR spectrum having peaks at 11.3, 104.8, 116.1, 132.5, 134.7 and 169.5±0.2 ppm;
c) a solid state ¹³C NMR spectrum having the following chemical shift absolute differences from a reference peak at 173.3 ppm±2 ppm of 162.0, 68.5, 57.2, 40.8, 38.6 and 3.8 ppm±0.1 ppm;
d) a solid-state ¹³C NMR spectrum substantially as depicted in FIGS. 8a, 8b or 8c; and
e) combinations of two or more of: a, b, c, or d.

5. The crystalline Cilofexor:Tromethamine according to claim 4, which is characterized by an X-ray powder diffraction pattern having peaks at 8.9, 12.3, 15.6, 18.7 and 20.7 degrees 2-theta±0.2 degrees 2-theta, and also having any one, two, three, or four additional peaks selected from 19.4, 23.5, 28.3 and 30.0 degrees 2-theta±0.2 degrees 2-theta; or which is characterized by an XRPD pattern having peaks at: 8.9, 12.3, 15.6, 18.7, 19.4, 20.7, 23.5, 28.3 and 30.0 degrees 2-theta±0.2 degrees 2-theta.

6. The crystalline Cilofexor:Tromethamine according to claim 4, which is further characterized by: an absence of peaks at 3.0-4.8 degrees 2-theta±0.2 degrees 2-theta; and/or an absence of peaks at 7.7-8.4 degrees 2-theta±0.2 degrees 2-theta or an X-ray powder diffraction pattern substantially as depicted in FIG. 3.

7. The crystalline Cilofexor:Tromethamine according to claim 1, which is an anhydrous form.

8. The crystalline Cilofexor:Tromethamine according to claim 1, wherein molar ratio of Cilofexor and Tromethamine is about 1:1.

9. The crystalline Cilofexor:Tromethamine according to claim 1, which contains: no more than about 20%, no more than about 10%, no more than about 5%, no more than about 2%, no more than about 1% or about 0% of any other crystalline forms of Cilofexor:Tromethamine.

10. The crystalline Cilofexor:Tromethamine according to claim 1, which contains: no more than about 20%, no more than about 10%, no more than about 5%, no more than about 2%, no more than about 1% or about 0% of amorphous Cilofexor:Tromethamine.

11. A pharmaceutical formulation comprising crystalline Cilofexor:Tromethamine according to claim 1.

12. (canceled)

13. The pharmaceutical formulation according to claim 11, further comprising at least one pharmaceutically acceptable excipient.

14. A process for preparing a pharmaceutical formulation, comprising combining the crystalline Cilofexor:Tromethamine according to claim 1 with at least one pharmaceutically acceptable excipient.

15. (canceled)

16. (canceled)

17. A method of treating a condition, the method comprising: administering a therapeutically effective amount of crystalline Cilofexor:Tromethamine according to claim 1 to a subject in need of treatment of the condition.

18. The method of treating according to claim 17, wherein the condition includes at least one of liver disease, Primary sclerosing cholangitis, Non-alcoholic fatty liver disease; Non-alcoholic steatohepatitis or Primary biliary cirrhosis.

19. (canceled)
